(19) United States
(12) Patent Application Publication
(10) Pub. No.: US 2024/0270787 A1
(43) Pub. Date: Aug. 15, 2024

(54) SEPARATION OF HYDROPHOBIC PROTEINS FROM MAMMALIAN HOST CELL PROTEINS

(71) Applicant: BAYER HEALTHCARE LLC, WHIPPANY, NJ (US)

(72) Inventors: HENDRI TJANDRA, UNION CITY, CA (US); XIAOYU PAN, SAN FRANCISCO, CA (US); RYAN ZOLYOMI, SAN JOSE, CA (US); DEEPA SURENDAR, SAN RAMON, CA (US)

(21) Appl. No.: 18/168,765

(22) Filed: Feb. 14, 2023

(51) Int. Cl. C07K 1/16; C07K 16/00

(52) U.S. Cl. CPC C07K 1/165; C07K 16/00

(57) ABSTRACT

What is described herein relates to a method for separating host cell proteins from proteins of interest, comprising the following steps
a) subjecting a mixture of host cell proteins and proteins of interest to at least a Protein A chromatography followed by a cation exchange chromatography (CEX) and up to two ion exchange chromatography (IEX) steps wherein one of the two ion exchange chromatography steps is a mixed mode chromatography (MM AEX/CEX) and the other one is an anion exchange membrane adsorber (MA AEX) in flow through mode OR
b) subjecting a mixture of host cell proteins and proteins of interest to non-ionic detergent during a Protein A chromatography wash step.

Removal or reduction of host cell proteins to a level which meets inter alia safety regulations is achieved during biotherapeutic isolation and purification. Most of the host cells proteins (HCPs) present, for example, in the cell culture expressing a monoclonal antibody can be typically removed during the affinity capture step e.g. via optimization of Protein A (ProA) process parameters. However, there are cases where a large quantity of host cell proteins is present and hence, a significant amount of HCPs still remains post-ProA processing step. In some cases, the host cell proteins interact strongly with the proteins of interests, and so the host cell proteins co-elute and/or co-purify with the biotherapeutic protein of interest. Therefore, there is a need for improved methods of separation of host cell proteins and biotherapeutic protein of interest.

This need is met by a method for separating host cell proteins from proteins of interest, comprising the following steps
a) subjecting a mixture of host cell proteins and proteins of interest to at least a protein A chromatography followed by a cation exchange chromatography (CEX) and two ion exchange chromatography (IEX) steps wherein one of the two ion exchange chromatography steps is a mixed mode chromatography (MM AEX/CEX) and the other one is a anion exchange membrane adsorber (MA AEX) in flow through mode OR
b) subjecting a mixture of host cell proteins and proteins of interest to non-ionic detergent during a protein A chromatography wash step.

To a skilled person it is clear that the described method is especially useful if a large quantity of host cell proteins is present and/or if host cell proteins interact strongly with the proteins of interests.

As used herein the term “detergent” means an amphipathic molecule that contains both hydrophobic and hydrophilic groups. These molecules contain a polar, hydrophilic moiety (‘head’) at the end of a long hydrophobic carbon chain (‘tail’).

The term “non-ionic detergent” means a detergent molecule that contains an uncharged hydrophilic head group(s). Examples of non-ionic detergents are Triton X-100, Triton X-112, Brij 35, Brij 58, Octyl b glucoside, Big CHAP, Deoxy Big CHAP, Tween 20 and Tween 80.

In a preferred embodiment of part a) of the method described herein the anion exchange membrane adsorber used in in flow through mode is a single use anion exchange membrane adsorber.

In a preferred embodiment of part a) of the method described herein the anion exchange membrane adsorber is a salt-tolerant single-use hydrophobic membrane absorber e.g. MA ST Polisher (3M, Maplewood, MN, USA) and MA STIC PA (Sartorius, Gottingen, Germany).

In a preferred embodiment a multi-modal anion exchanger (MM AEX) is used as anion mixed mode chromatography resin such as the Capto adhere (Cytiva, Marlborough, MA, USA).

In a preferred embodiment of part b) of the method described herein the non-ionic detergent is selected from the group consisting of Triton X-100 (MilliporeSigma, Burlington, MA, USA), Tween20 and Tween80 (Avantor, Radnor Township, PA, USA).

In an especially preferred embodiment of part b) of the method described herein, the non-ionic detergent is selected from the group consisting of Triton X-100, Tween 20 and Tween 80 and applied at a range of between 0.5 and 4.0% [w/w].

Most preferably the non-ionic detergent is Triton X-100 in a range of between 0.5-3.5% [w/w].

In a further especially preferred embodiment of part b) of the method described herein after subjecting the mixture of host cell proteins and proteins of interest to non-ionic detergent during a protein A chromatography wash step the mixture is also subjected to a CEX and a MA AEX.

The host cell protein (HCP) content can be determined by methods known in the art, for example via using Cygnus CHO HCP 3rd Generation F550. A standard curve can be constructed using four-parameter logistic fit. Alternatively, HCP levels can be quantified using CISBIO CHO HCP Catalog #6FHCPPEG. The plate could be read on PHERAstar microplate reader, BMG Labtech Inc., (Durham, NC), connected to a computer with the PHERAstar Installation Package 3.10 R4 or above for Windows, containing Control software version 3.10 R3 and MARS version 2.10.R3 or above. The concentration of the HCP species in the sample(s) is interpolated from a HCP standard curve using a four parameter fit and regression analysis, and is reported in ppm or μg HCP per mg of antibody/protein of interests.

In a preferred embodiment of part b) of the method described herein the hydrophobicity of the protein of interest is determined using HIC-HPLC and/or RPLC.

Hydrophobic interaction chromatography (HIC) separates molecules based on their hydrophobicity. HIC is a useful separation technique for purifying proteins while maintaining biological activity due to the use of conditions and matrices that operate under less denaturing conditions.

In a preferred embodiment of part b) of the method described herein, the hydrophobicity of the protein of interest is determined using HIC-HPLC. Depending on the outcome of this HIC-HPLC the protein of interest is classified as less hydrophobic or hydrophobic either based on comparison to a reference protein or based on absolute values.

As already mentioned above it is clear to a skilled person, that the value determined via HIC-HPLC for the hydrophobicity of a given protein of interest can be an empirical and/or relative value and/or an absolute value. In other words, if the protein of interest is classified as less hydrophobic or more hydrophobic based on comparison to a reference protein then the behavior of the reference protein can be directly compared to the behavior of the protein of interest under comparable HIC-HPLC conditions i.e. where the HIC-HPLC main peak retention time of the protein of interest is relative to that of the reference protein. This analysis can determine if a protein is more hydrophobic or less hydrophobic than the reference protein.

Preferably a given protein of interest is classified as hydrophobic if the retention time of the protein of interest is ≥retention time of antibody G disclosed as TPP9252 in WO2020/089380 A1 (Reversal Agents for neutralizing the therapeutic activity of anti-FXIa antibodies) under comparable HIC-HPLC conditions.

Alternatively, the absolute values of the HIC-HPLC main peak retention time of the reference protein and the protein of interest can be compared. Moreover, empirical values could be used to classify a protein of interest.

In case the protein of interest is determined to be hydrophobic in a preferred embodiment of part b) of the method described herein, it is preferred that the wash buffer comprising the non-ionic detergent during the Protein A chromatography wash step has a pH in the range of 4.8-7.4.

In case the protein of interest is determined to be hydrophobic in a preferred embodiment of part b) of the method described herein, it is especially preferred that in case the protein of interest is determined to be hydrophobic the wash buffer comprising the non-ionic detergent during the Protein A chromatography wash step has a pH of 5.5.

In addition to employing a non-ionic detergent during the Protein A chromatography wash step at a pH in the range of 5.2-7.4 in case the protein of interest is determined to be hydrophobic in an alternative embodiment of part b) of the method described herein two protein A wash steps using non-ionic detergents can be performed, wherein one Protein A wash step is carried out with a buffer with a pH in the range of 7.0-7.4 and one Protein A wash step is carried out with a buffer with a pH in the range of 5.2-5.8.

In a preferred embodiment of the method described herein the protein of interest is expressed in a mammalian cell culture and is a monoclonal antibody.

In an especially preferred embodiment of the method described herein the mammalian cell culture is a Chinese hamster ovary cell culture.

Generally a setting where the method described herein can be employed is an antibody generation and purification process described in the following. The process described is suitable for a wide range of monoclonal IgG antibodies and is optimized for bioreactor titers up to 8 g/L with an expected harvest volume of e.g. 2000 L from a single-use or stainless-steel bioreactor. With appropriate modifications, antibody cultures with other titer ranges can be processed.

Following thawing the CHO cells are transferred to shake flasks and cultured for 3-8 passages i.e. a maximum of 25 days in expansion culture e.g. in a shake flaks with a flask volume of up 2.8 l, a culture volume of 1.6 l at a temperature of around 36.5° C. and a pH of around 7.0. Optional pre-culture cultivation (N−2) is carried out in batch mode for 3-8 days using a single-use or stainless steel bioreactor with a size of up to 200 kg, a culture volume of up to 50 kg a temperature of around 36.5° C. a pH of around 7.0 and a pO2 preferably of around 40% using 0.5×10⁶ cells/ml-0.7×10⁶ cells/ml as seeding cell density. The N−1 pre-culture cultivation is carried out in batch mode for 3-6 days using a single-use or stainless steel fermenter with a fermenter size of up to 200 kg, a culture volume of up to 200 kg, a temperature of around 36.5° C. a pH of around 7.0 and a preferred pO2 of around 40% using 0.5×10⁶ cells/ml-1.0×10⁶ cells/ml as seeding cell density. Alternatively N−2 and N−1 cultivations are both carried out in one expansion bioreactor, preferably a single use bioreactor with a maximum working volume of 200 L e.g. a XDR200 reactor. This method includes a fill up step after 2 to 4 days, a culture volume of up to 200 kg, a temperature of around 36.5° C. a pH of around 7.0 and a pO2 preferably of around 40% using 0.5×10⁶ cells/ml-1.6×10⁶ cells/ml as seeding cell density. Production cultivation is carried out with 0.5×10⁶ cells/ml-1.5×10⁶ cells/ml as seeding cell density in fed batch mode for 10-21 days using a single-use or stainless steel fermenter with a fermenter size of up to 2500 kg and a culture volume of up to 2500 kg, a temperature of around 36.5° C. a pH of around 7.0 and preferably a pO2 of around 40%. In the state of the art maintaining pO2 of the cell culture during a production phase in fermentation/cultivation is disclosed e.g. via air-sparging. During production cultivation the target final glucose concentration of the cell culture is 6-9 g/l which can be achieved via continuous addition or daily addition of 1-4 doses. Moreover, in process control is used to monitor metabolites such as lactate, glutamine and glutamate and defoamer is added if necessary either continuously or in individual doses. Cell culture temperature in the production bioreactor is cooled down to 15° C. at the time of harvest.

The cell culture is harvested via a combination of depth filtration and 0.2 μm sterile filtration or employing a hybrid purifier containing a Q-functional anion exchange media. Alternatively harvesting via centrifugation or ATF filtration could be envisaged. Clarified harvest is stored e.g. in stirred stainless-steel storage tank(s). In the state of the art cooling of the clarified harvest as well as storage in single-use bags with maintenance of a head space is disclosed.

During further purification closed processing and single-use systems are preferred: stainless-steel skids or systems are also possible with appropriate cleaning and change-over procedures. Single-use bags are typically of low-density or ultra-low density polyethylene as contact layer. Among others. Sodium Acetate/Acetic Acid or Tris/Tris-HCl buffer systems may be used, usually in a total concentration of 50 mM and including 50 mM NaCl, unless otherwise noted. Buffers and process intermediates of the respective unit operations (e.g. Load. Eluate or Flowthrough) are usually 0.2 μm filtered inline or with a filtration assembly, using e.g. PES membrane and/or fleece filters. These solutions are usually stored in single-use bags or stainless-steel vessels, at ambient (e.g. 18-26° C.) or 2-8° C. conditions.

### Capture

Antibody capture is performed using Protein A-based chromatography, utilizing e.g. Cytiva MabSelect SuRe, MabSelect SuRe LX, Mab Select PrismA or Purolite A50 resins and Purolite Praesto Jetted A50 resins. Loading is conducted following clarification, with or without pre-concentration by ultrafiltration. The loading density is typically 45-75 g/L, using a single or a variable flow rate to apply the clarified harvest onto the column (120-400 cm/h). The chromatography can be performed in batch mode with one or more columns, or in continuous (MCC (multi-column chromatography)/SMB (simulated moving bed)) mode with up to 8 columns. Pre- or self-packed columns are used with a typical bed height of 15-20 cm for batch mode. For continuous mode, a contact time of the clarified harvest to the resin of 1-4 min is targeted. The capture is performed in one or multiple cycles depending on the antibody titer. Prior to loading, the column is equilibrated using a Tris buffer (e.g. pH 7): washing is performed with at least two washing steps: high NaCl concentration in the first wash buffer, then low NaCl concentration in the second buffer (e.g. 1st wash: 1 M NaCl, pH 5.5; 2nd wash: 50 mM NaCl, pH 6.0) in acetate buffer. Elution may for instance be achieved by low pH (e.g. 3.5) acetate buffer containing 0-50 mM NaCl in typically <5 column volumes (CV) with UV- or volume-controlled eluate collection. The eluated may be pooled (including mixing) at this step or after virus inactivation. The affinity column is regenerated by >3 CV acetic acid (e.g. 0.1 M, incl. 500 mM NaCl), sanitization (clean-in-place, CIP) occurs with 0.11.0 M NaOH for 30 min, re-equilibration with >4 CV. The column is stored in e.g. 20% EtOH or 2% BnOH.

Alternatively, affinity capture from clarified harvest can be performed with affinity ligands on a cellulose architecture (e.g. Fibro Prism, CytivaA) using the same loading schemes and buffer system as outlined above. Regeneration or CIP block may be skipped for optimal processing time. A contact time of 5-20 seconds can be applied to Fibro units of varying sizes (e.g. 0.4 mL to 2.4 L). Typically, a loading capacity of 25-35 g/L unit volume can be realized. A single unit can be used to process a single batch or multiple batches, depending on the Fibro unit and bioreactor sizes as well as titer of antibody culture. Affinity loading materials can be further clarified by charged filters at >300 L/m2 throughput prior to loading to maximize lifetime use and to minimize fouling.

### Low-pH Virus Inactivation

The capture eluate is adjusted with HOAc or HCl to pH 3.7-3.9 and held for 120-240 min in the same or a separate bag at >18° C. to inactivate potentially present viruses. Alternatively, viral inactivation is carried out at pH 3.4-3.6 for >30 minutes. The process intermediate is then adjusted to storage or to further processing conditions, e.g. to pH 4-7 using 1-2 M Tris/Tris-HCl titrant stock.

### Polishing 1

The antibody is further polished by anion exchange chromatography (AEX), typically in the form of membrane capsule or cassettes (e.g. Sartorius Sartobind STIC PA 4 mm or 3M™ Polisher ST) in flow through mode, with flow rates <350 MV/h, for removal of process-related and/or product-related impurities and particles. The entire amount from one batch may be processed in one or in multiple cycles, depending on the antibody amount. Prior to loading, the intermediate is adjusted to a target conductivity of e.g. 8-20 mS/cm, and a target pH of e.g. 7.0 for antibodies with basic isoelectric points. For antibodies with an isoelectric point lower than pH 7.0, the conductivity could be 5-20 mS/cm, and the pH can be between its isoelectric point and pH 5.0 The membrane is equilibrated with Tris buffer, pH 7.0 or Acetate buffer pH 5-6, and loaded with densities between typically 0.5-10 g antibody/mL membrane volume (MV). A chase with equilibration buffer may be performed and combined with the collected flow-through, the collection criteria of which is controlled by e.g. UV or volume. The membrane adsorber may be used once or multiple times, for which it is regenerated (1 M NaCl for 25 MV) and, re-equilibrated (Tris buffer pH 7.0).

### Polishing 2

Final polishing may for instance be achieved by cation exchange chromatography (CEX). In principle, the order of AEX and CEX unit operations may be reversed. The CEX unit operation is usually performed in bind & elute mode with flow rates between 100-300 cm/h. Pre- or self-packed columns with resins e.g. Cytiva Capto S ImpAct or Capto SP ImpRes or Purolite Praesto Jetted XS50 may be used at 15-20 cm bed height. The polishing step is performed in one or multiple cycles depending on the antibody amount. If needed, the load may be adjusted to target conductivities between 5-9 mS/cm and to pH 4.5-7.5 with HOAc, Tris titrants or WFI.

The load is applied at typical densities between 40-105 g/L onto a CEX column equilibrated with >5 CV acetate buffer (pH and conductivity matched to CEX load) and washed afterwards using >5 CV of the same buffer. The antibody is eluted using an acetate buffer with suitable NaCl concentration (>50 mM: <500 mM), and the eluate is collected by UV-control in typically <10 CV collected eluate volume. The column is regenerated using 0.5-2 M NaCl in acetate buffer, re-equilibrated (>5 CV with CEX equilibration buffer), sanitized (0.5-1.0 M NaOH for >30 min), re-equilibrated (>5 CV with CEX equilibration buffer), and finally stored in e.g. 20% EtOH or 2% BnOH, with or without 200 mM NaOAc/HOAc.

### Additional Polishing

When additional polishing is needed to remove residual product- or process-related impurities, a mixed mode chromatography (MMC) may be used in place of, or additional to, the CEX step. Example of mixed mode chromatographic resins are Cytiva Capto adhere and Capto MMC ImpRes. This unit operation is typically operated in a flow-through mode but bind-and-elute mode may also be employed. The column can be self- or pre-packed with a bed height of 5-20 cm at various diameters and operated at flow rates between 100-500 cm/h in a flowthrough mode or 100-220 cm/hr in a bind-and-elute mode.

The load is typically adjusted, if needed, to target conductivities between 3-12 mS/cm and to pH 4.2-7.5 with HOAc, Tris titrants or WFI The load is applied at typical densities between 75-300 g/L onto an MMC column equilibrated with >5 CV acetate buffer (pH and conductivity matched to MMC load). A chase with equilibration buffer may be performed and combined with the collected flow-through, the collection criteria of which is controlled by e.g. UV or volume. The MMC column might be used once or multiple times depending on the amount of antibody to be polished. The column is regenerated using 0.5-2 M NaCl in acetate buffer, re-equilibrated (>5 CV with equilibration buffer), sanitized (0.5-1.0 M NaOH for >30 min), and finally stored in e.g. 20% EtOH or 2% BnOH.

### Nanofiltration

Potentially remaining adventitious viruses are removed by PDVF-based nanofiltration, e.g. using Planova BioEX with or without prefilter. The load is applied at constant pressure (e.g. 2.0-3.4 bar) with a loading density of 1500-5000 g antibody/m2 to a pre-equilibrated nanofilter (>5 L/m2 acetate buffer, recipe e.g. matched to MA AEX or CEX elution conditions). The filter may be chased using the same buffer and the integrity of the filter is typically tested post-use. Upon failure of the integrity test, this step may be repeated. Alternatively, adventitious viruses can be removed by cellulose-based nanofiltration with or without a prefilter. The load is applied at a constant pressure (e.g. 0.8-1.2 bar) with a loading density of 500-2000 g antibody/m2 to a pre-equilibrated nanofilter (>5 L/m2 acetate buffer, recipe e.g. matched to MA AEX or CEX elution conditions).

### Concentration and Buffer Exchange

The intermediate is concentrated by ultrafiltration to 15-110 g/L, diafiltered with >6 diafilter volumes against a diafiltration buffer (DFB) (e.g. 10 mM histidine, 10 mM methionine, 30 mM arginine, pH 5.3), and, if needed, further concentrated up to 200 g/L. This is typically performed using a tangential-flow filtration device and a suitable membrane with a cutoff of 30-50 kDa (e.g. Millipore Pellicon, Sartorius Hydrosart, Pall Omega) with a load of typically 300-1000 g antibody/m2. The process is typically controlled by feed or retentate flow (e.g. 2-7 L/min/m2), alternatively by feed pressure (2-4 bar), and by TMP (0.7-2 bar). The membrane may be chased by DFB and the pooled retentate diluted with DFB to the target concentration. The TFF system and membranes are sanitized with 0.5 M NaOH and equilibrated with acetate buffer or DFB. The membrane integrity or permeability is tested pre-use.

### Stabilization

In an optional stabilization step, an excipient concentrate is added to produce bulk drug substance (BDS). A bioburden reduction filtration (e.g. 0.2 μm) is performed using an e.g. pre-equilibrated filter (e.g. excipient concentrate diluted in DFB), which is tested post-use for integrity. Upon failure of the integrity test, the filtration step may be repeated. Alternatively both the bulk drug substance and the excipient concentrate are subjected to bioburden reduction filtration (e.g. 0.2 μm) prior to joining the excipient concentrate and the bulk drug substance aseptically.

### Fill & Freeze

The BDS is filled in suitable bags with an LDPE contact layer (e.g. 5 or 10 L) which may be connected to individual i.d. sampling compartments that are preferably aseptically connected and disconnected.

The bags are individually protective packaged by optional vacu-sealing (LDPE overwrap bags) and placed in protective shells (e.g. ROSS Shells). After an optional intermediate 2-8° C. storage, the shelled bags are frozen (using a plate- or a passive freezer) and subsequently stored at ≤−30° C. or ≤−70° C., e.g. +/−10° ° C., alternatively at ≤−25 or ≤−60° C., e.g. +/−5° C.

### EXAMPLES

### Example 1 Increased Presence of Host Cell Proteins after Monoclonal Antibody Purification

During routine process development of monoclonal antibody ‘A’ it was found that the host cell protein concentration was higher than expected from purifications of other monoclonal antibodies (cf. Table 1). The host cell protein concentration was determined using Cygnus CHO HCP 3rd Generation F550. The standard curve was constructed using four-parameter logistic fit.

TABLE 1

| | | Reference |
|---|---|---|
| | mAb ‘A’ | mAb ‘B’ |
| Host cell protein [ppm] after Protein A | ~5000-9000 | ~200-2200 |
| and cation exchange chromatography | | |

### Example 2 Establishing a Non-Ionic Detergent Wash Step

In order to remove additional host cell protein a cell culture fluid comprising monoclonal antibody A as well as host cell proteins was subjected to Protein A chromatography using a MabSelect SuRe LX, (Cytiva/GE Healthcare) resin. The wash buffer containing 1 M sodium chloride and 50 mM sodium acetate at pH 5.2 was supplemented with Triton X-100, Tween-20 or Tween-80 at different concentrations [cf. Table 2]. The host cell protein level was reduced greatly compared to that of the control with Triton X being the most effective non-ionic detergent tested, which was also confirmed in another experiment (data not shown). For this particular antibody Triton X in a range of between 1-1.5% [w/w] was found to be most effective.

| Added non-ionic detergent | Host cell protein content in ppm |
|---|---|
| Control: no detergent | 28.843 |
| Triton X 1% | 3.117 |
| Triton X 2% | 2.028 |
| Triton X 3% | 1.492 |
| Tween 20 1% | 9.548 |
| Tween 20 2% | 9.805 |
| Tween 20 3% | 6.758 |
| Tween 80 1% | 19.506 |
| Tween 80 2% | 20.235 |
| Tween 80 3% | 16.167 |

### Example 3 Additional Anion Exchange Chromatography Step

200 liters of antibody A cell culture was clarified by depth filtration. A volume of the clarified cell culture fluid at a titer of 2.4 g/L was subjected to Protein A-based chromatography using a 20-cm bed height of packed Cytiva MabSelect SuRE resin at a loading density of 52 g/L. A flow rate of 120-400 cm/hr was applied to the antibody ‘A’ comprising cell culture fluid for variable loading it onto the column. Prior to loading, the column was equilibrated using a Tris buffer (pH 7): washing was performed with two Wash buffers sequentially. The first wash was with 1 M NaCl, pH 5.5, and the second wash was with 50 mM NaCl, pH 6.0, both buffered with acetate ions in acetate buffer. The antibody was eluted using a pH 3.4 acetate buffer.

The captured eluate was adjusted with acetic acid to pH 3.7-3.9 and held for 120-240 min at temperature of 18° C. or higher to inactivate potentially adventitious viruses. The process intermediate was then adjusted (“quenched”) to pH 5.0 using 1 M Tris-HCl titrant stock The host cell protein concentration was determined via Elisa or HTRF to be 17 356 ppm.

In the next processing step a cation exchange chromatography (CEX) was performed. It is clear to a skilled person that in principle the sequence order of AEX and CEX unit operations may be reversed. Here the CEX load was applied to a 20-cm bed height Capto S ImpAct at a flow rate of 200 cm/h and a conductivity of 4.5 mS/cm in a bind-and-elute mode. The load was applied at a density of not more than (NMT) 80 g per L resin onto the CEX column equilibrated with >5 CV acetate buffer (pH 5.0±0.2 and conductivity 5.3±0.3 mS/cm) and washed afterwards using >5 CV of the same buffer. The antibody was eluted using an acetate buffer with a suitable NaCl concentration (e.g. 130 mM). The host cell protein concentration was determined via ELISA or HTRF to be 2 119 ppm

In the next step anion exchange chromatography (AEX), using a Sartobind STIC PA membrane adsorber (MA) in a flow-through mode, with a flow rate of NMT 10 membrane volume per minute. A target conductivity of 5 mS/cm or 9 mS/cm and a target pH of 7.0 was performed. The membrane was equilibrated with a Tris buffer pH 7, conductivity of 5-9 mS/cm and loaded with a loading density of 2 gramme of antibody per mL membrane volume.

The host cell protein concentration was determined via ELISA or HTRF to be 45 or 142 ppm under the 5 mS/cm or 9 mS/cm loading conductivity, respectively.

The antibody was further polished by an anion exchange mixed mode (MM AEX) chromatography step in a flow-through mode. In this third polishing step, the MA AEX Flowthrough from the second polishing step was applied to a Capto adhere resin. The column was equilibrated and chased with the same buffer. Both the Flowthrough and Chase fractions were pooled. The host cell protein concentration was determined as given in Table 3 below.

TABLE 3

| | Conductivity, 9 mS/cm | Conductivity, 5 mS/cm |
|---|---|---|
| | HCP in ppm | HCP in ppm |
| Prior to CEX | 17356 | 17356 |
| Prior to AEX | 2119 | 2119 |
| After AEX | 142 | 445 |
| After 2^{nd} AEX | 31 | 14 |

The experiment was repeated changing the order of 3M MA ST Polisher and Capto adhere which also led to significant reduction of host cell proteins.

1. Method for separating host cell proteins from proteins of interest, comprising the following steps
a) subjecting a mixture of host cell proteins and proteins of interest to at least a protein A chromatography followed by a cation exchange chromatography (CEX) and up to two ion exchange chromatography (IEX) steps wherein one of the two ion exchange chromatography steps is a mixed mode chromatography (MM AEX/CEX) and the other one is an anion exchange membrane adsorber (MA AEX) in flow through mode OR
b) subjecting a mixture of host cell proteins and proteins of interest to non-ionic detergent during a protein A chromatography wash step.

2. Method according to claim 1 part a) wherein anion exchange chromatography in flow-through mode is preformed using a salt-tolerant hydrophobic the single use membrane absorber e.g. MA ST Polisher (3M, Maplewood, MN, USA) and MA STIC PA (Sartorius, Gottingen, Germany).

3. Method according to claim 1 part a) wherein a multi-modal anion exchanger (MM AEX) is used as anion mixed mode chromatography resin such as the Capto adhere (Cytiva, Marlborough, MA, USA).

4. Method according to claim 1 part b) wherein the non-ionic detergent is selected from the group consisting of Triton X100, Tween20 and Tween80.

5. Method according to claim 4 wherein the non-ionic detergent is selected from the group consisting of Triton X100, Tween20 and Tween80 and applied at a range of between 0.5 and 3.5% [w/w].

6. Method according to claim 1 part b) wherein after subjecting the mixture of host cell proteins and proteins of interest to non-ionic detergent during a protein A chromatography wash step the mixture is also subjected to a CEX and a MA AEX.

7. Method according to claim 1 part b) wherein the hydrophobicity of the protein of interest is determined using HIC-HPLC and/or RPLC and in case the protein of interest is determined to be hydrophobic the wash buffer comprising the non-ionic detergent during the Protein A chromatography wash step has a pH in the range of 5.2-7.4 even more preferred a pH of 5.5.
